(19) United States
(12) Patent Application Publication
(10) Pub. No.: US 2004/0002683 A1
(43) Pub. Date: Jan. 1, 2004

(54) Percutaneous medical insertion device

(76) Inventors: Thomas J. Nicholson, York, PA (US); William Jay Nicholson, York, PA (US); W. Jay Nicholson, York, PA (US)

Correspondence Address: Jennifer M. Slonaker, Esquire, Barley, Snyder, Senft & Cohen, LLC, 126 East King Street, Lancaster, PA, 17602, US

(21) Appl. No.: 10/319,311

(22) Filed: Dec. 12, 2002

Related U.S. Application Data
(63) Continuation-in-part of application No. 10/180,758, filed on Jun. 26, 2002.

(51) Int. Cl. A61M 5/178

(52) U.S. Cl. 604/164.01

(57) ABSTRACT

A percutaneous medical insertion device for positioning a tubular member or a catheter within an interior cavity or a vein. The catheter having a flexible end portion and an opening that removeably receives a needle. The flexible end portion is essentially straight when the needle is positioned in the flexible end portion and the flexible end portion essentially curls when the needle is removed from the flexible end portion. The catheter has an opening that allows for the creation of a ninety degree angle of the catheter and simultaneously allowing a needle to be inserted through the flexible end of the catheter. After insertion into the interior cavity or the vein, the needle is removed from the catheter. The flexible end portion of the catheter curls when the needle is removed from the catheter to protect the interior of the interior cavity or the vein as the catheter is advanced.

### FIELD OF THE INVENTION

The invention relates to a percutaneous medical insertion device and, more particularly, to a percutaneous medical insertion device for positioning a catheter or tubular member in an interior cavity or vein.

### BACKGROUND OF THE INVENTION

Percutaneous medical insertion devices are commonly used to place central lines or catheters in veins or to form a passageway in a tracheal wall or skin tissue for insertion of a tubular member. For example, percutaneous medical insertion devices are commonly used for placing special intravenous (IV) catheters in a central vein, such as a subclavian internal jugular, or femoral vein, to allow for administration of large volumes of fluid, supplemental nutrition, blood draws, and pressure measurement. Percutaneous medical insertion devices are also commonly used to provide an air passageway when a patient's airway is obstructed by forming a passageway in the patient's tracheal wall for insertion of a tracheal tube. Other common usages for percutaneous medical insertion devices include the formation of passageways for the placement of chest tubes, gastrectomy tubes, trocars for laprascopic surgery and intra-abdominal drains.

One common example of a percutaneous medical insertion device and method used for forming a passageway in a tracheal wall is disclosed in U.S. Pat. No. 5,653,230. In U.S. Pat. No. 5,653,230, a slight incision is made with a scalpel through a patient's skin adjacent to the tracheal wall. A hollow needle is inserted through the incision and the tracheal wall until it is received in a tracheal passage. A wire guide is inserted into the hollow needle until it is received in the tracheal passage. The hollow needle is then withdrawn, leaving the wire guide inside the tracheal passage. A catheter attached to a dilator tube and provided with an inflatable balloon member is positioned over the wire guide. The catheter is advanced along the wire guide until the inflatable balloon member is received in the tracheal wall. A dilator is then activated providing a fluid supply through the dilator tube and into the inflatable balloon member. The inflatable balloon member expands to dilate a portion of the tracheal wall. The inflatable balloon member is then deflated by extracting the liquid to leave a passageway. A tracheal tube disposed on the dilator tube is then advanced along the catheter and received in the passageway. As the tracheal tube is received in the passageway, the deflated balloon member is advanced into the tracheal passage. The deflated balloon member and wire guide are then withdrawn from the tracheal tube, leaving the tracheal tube in the passageway.

The formation of a passageway by the conventional percutaneous medical insertion device and method requires a significant number of intricate steps. Specifically, the known device and method requires an incision before insertion of the needle and the advancement of a wire guide before insertion of the catheter. The number and intricacy of the steps not only leaves room for error, but also the length of time needed to perform all of the steps is poorly suited to an emergency situation where prompt restoration of the patient's air passageway is critical.

Similarly, the percutaneous medical insertion device and method of placing a catheter in a vein for the administration of fluids requires several intricate steps. A hollow needle is inserted through a patient's skin and into a vein. A wire guide is inserted into the hollow needle until it is received in the vein. A skin incision is made over the wire guide. Dilation is made over the wire guide. A catheter is advanced over the wire guide until the catheter is received in the vein. The wire guide is then withdrawn leaving the catheter in the vein.

The positioning of a catheter in a vein by the conventional percutaneous medical insertion device and method requires a significant number of intricate steps. Specifically, the known device and method requires advancement of a wire guide before insertion of the catheter. The number and intricacy of the steps not only leaves room for error, but adds to the common risks associated with catheter placement, which include pneumothorax, bleeding, and infection.

It is therefore desirable to develop a percutaneous medical insertion device and method that can atraumatically place a catheter into a vein or passageway to allow for the administration of fluids or to form a passageway for a tubular member in an integral step that eliminates the use of a wire guide to eliminate error and conserve time.

### SUMMARY OF THE INVENTION

The invention relates to a percutaneous medical insertion device for positioning a tubular member or a catheter within an interior cavity or a vein. The catheter having a flexible end portion and an opening that removeably receives a needle. The flexible end portion is essentially straight when the needle is positioned in the flexible end portion and the flexible end portion essentially curls when the needle is removed from the flexible end portion.

The invention further relates to a percutaneous method for positioning a catheter or tubular member in the body. A tip end of a needle removeably disposed in a catheter is percutaneously inserted through a wall of an interior cavity or a vein and into the interior cavity or the vein. The needle is withdrawn from the catheter leaving a flexible end portion of the catheter disposed in the interior cavity or the vein. The flexible end portion of the catheter curls when the needle is removed from the catheter to protect the interior of the interior cavity or the vein as the catheter is advanced.

### BRIEF DESCRIPTION OF THE DRAWINGS

FIG. 1 is a cross-sectional view of a first embodiment of a percutaneous medical insertion device.

FIG. 2 is a side view of a catheter of the device of FIG. 1.

FIG. 3 is a side view of a needle of the device of FIG. 1.

FIG. 4 shows the insertion of the device of FIG. 1 into a tracheal passage.

FIG. 5 shows a flexible end portion of the catheter curled in an essentially j-shape in the tracheal passage after the needle is withdrawn from the catheter.

FIG. 6 shows advancement of the flexible end portion of the catheter curled in an essentially j-shape in the tracheal passage.

FIG. 7 shows an inflatable balloon member received in a tracheal wall.

FIG. 8 shows advancement of a tubular member into a passageway in the tracheal wall formed by the inflatable balloon member.

FIG. 9 shows the tubular member positioned in the passageway before the catheter is withdrawn.

FIG. 10 shows the tubular member positioned in the passageway after the catheter is withdrawn.

FIG. 11 is a cross-sectional view of a second embodiment of the percutaneous medical insertion device.

FIG. 12 is a side view of a catheter of the device of FIG. 11.

FIG. 13 shows insertion of the device of FIG. 11 into a vein.

FIG. 14 shows advancement of a flexible end portion of the catheter curled in an essentially j-shape in the vein.

FIG. 15 shows the catheter positioned in the vein after the needle is withdrawn.

### DETAILED DESCRIPTION OF THE INVENTION

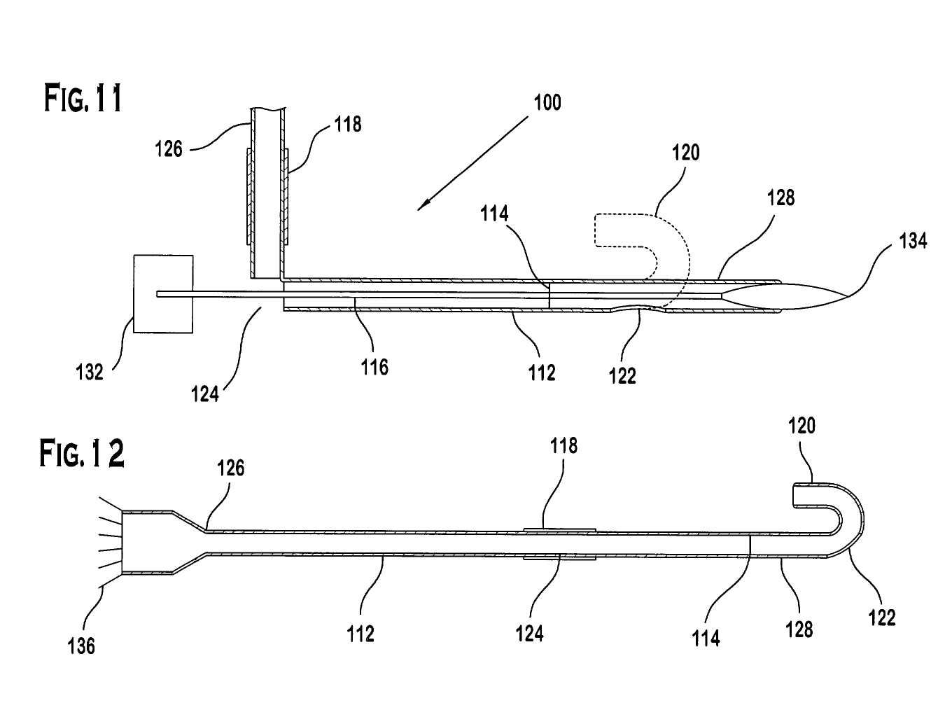

A first embodiment of the percutaneous medical insertion device 10 will first be described with reference to FIGS. 1-10. A second embodiment of the percutaneous medical insertion device 100 will then be described with reference to FIGS. 11-15.

FIG. 1 shows the first embodiment of the percutaneous medical insertion device 10. The device 10 comprises a catheter 12, a tubular member 18 and a needle 16. The needle 16 is removeably positioned within the catheter 12. The catheter 12 has an inflatable balloon member 14 and a first catheter end 26 having an attachment part 20 and a second catheter end 28 having a flexible end portion 32 that curls about the catheter 12 in an essentially j-shape (shown in phantom) when the needle 16 is removed. The tubular member 18 is positioned about the catheter 12.

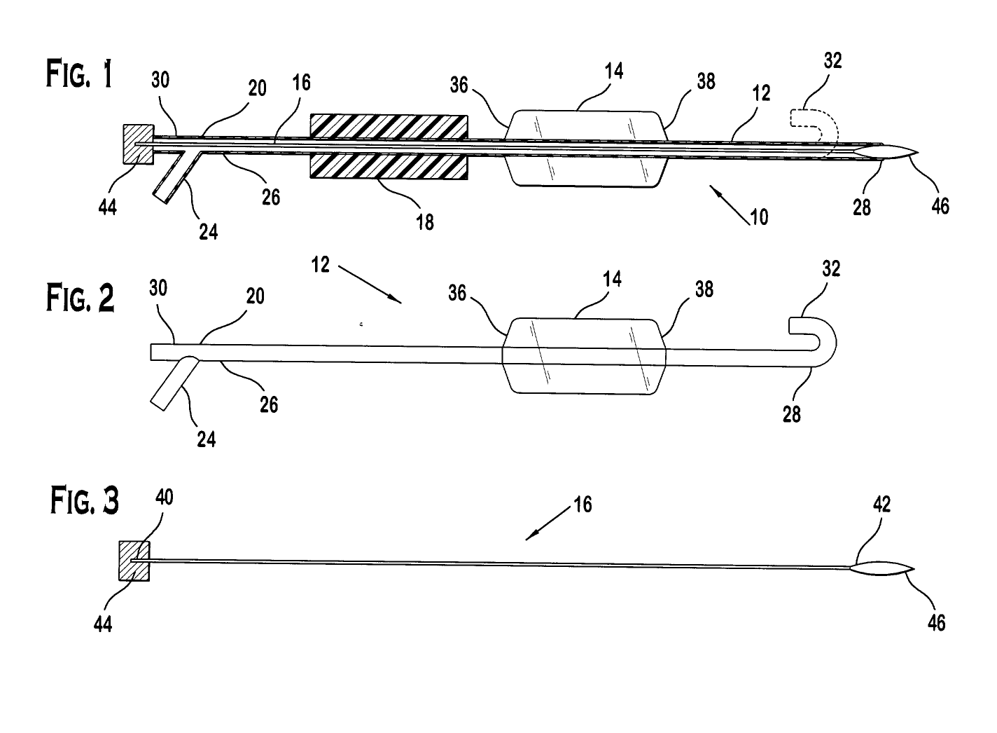

The individual components of the device 10 will now be described in greater detail. Shown in FIG. 2, the catheter 12 comprises a flexible hollow tubular member extending from the first catheter end 26 to the second catheter end 28. The first catheter end 26 is provided with the attachment part 20. The attachment part 20 has a needle attachment portion 30 and a dilator attachment portion 24. The attachment part 20 may be integrally formed with the catheter 12 or may be a separate piece attached to the first catheter end 26. The second catheter end 28 has a flexible atraumatic end portion 32. The flexible end portion 32 essentially curls about the catheter 12 in an essentially j-shape. The length of the catheter 12 may vary depending upon the application, but a preferred length of the catheter 12 is essentially 17 cm.

Proximate the flexible end portion 32 is the inflatable balloon member 14. The inflatable balloon member 14 has an inflated periphery substantially similar to the size of the desired passageway. The inflatable balloon member 14 is essentially cylindrical in shape and has a first balloon end 36 and a second balloon end 38. The first balloon end 36 and the second balloon end 38 are tapered at an acute angle with respect to the longitudinal axis of the catheter 12. The inflatable balloon member 14 measures essentially 3 cm from the first balloon end 36 to the second balloon end 38. The first balloon end 36 is preferably positioned essentially 8 cm from the first catheter end 26 to ensure sufficient space between the dilator attachment portion 24 and the inflatable balloon member 14 for operation of a conventional dilator (not shown). The second balloon end 38 is preferably positioned essentially 6 cm from the second catheter end 28 to ensure proper advancement of the second catheter end 28 for positioning of the inflatable balloon member 14. It will be appreciated and understood by those in the art, however, that the dimensions and the positioning of the inflatable balloon member 14 may vary depending upon the desired size of the passageway and the medical application to obtain substantially similar results.

Shown in FIGS. 1 and 3, the needle 16 may be a conventional surgical needle having a diameter smaller than the internal diameter of the catheter 12. The needle 16 has a first needle end 40 and a second needle end 42. The first needle end 40 has an attachment receiving portion 44 for engagement with the needle attachment portion 30 of the attachment part 20 of the catheter 12. The second needle end 42 is formed to have an essentially pointed tip 46. The length of the needle 16 is such that the pointed tip 46 extends beyond the second catheter end 28 when the needle is removeably received in the catheter 12.

Shown in FIG. 1, the tubular member 18 may be a conventional medical tube suitable for use in medical applications, such as a tracheal tube or a chest tube. The tubular member 18 has an external diameter slightly smaller than the periphery of the inflated balloon member 14, and an internal diameter larger than the external diameter of the catheter tube 12. The tubular member 18 is positioned about the catheter 12 proximate the first catheter end 26 and adjacent to the first balloon end 36.

A method for forming a passageway for insertion of a tubular member using the device 10 will now be described with reference to FIGS. 4 through 10. Although the device 10 is illustrated as forming a passageway in a tracheal wall 50 for insertion of tubular member 18 in the form of a tracheal tube, it will be understood and appreciated by those skilled in the art that the device 10 may be used in a wide variety of medical applications and in conjunction with other body cavities.

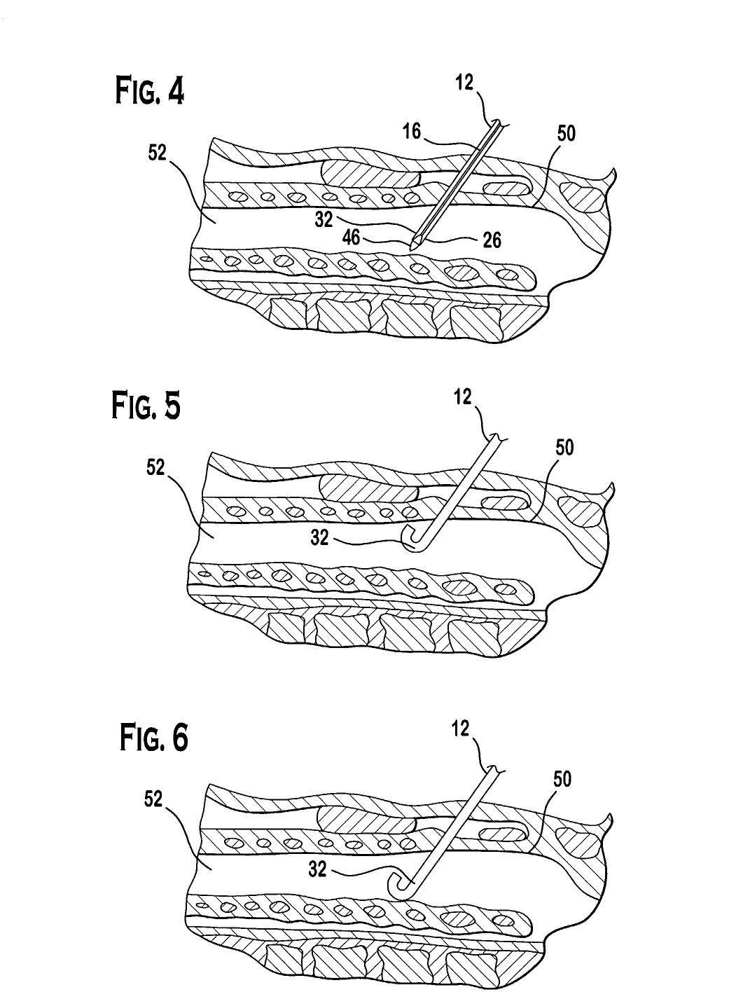

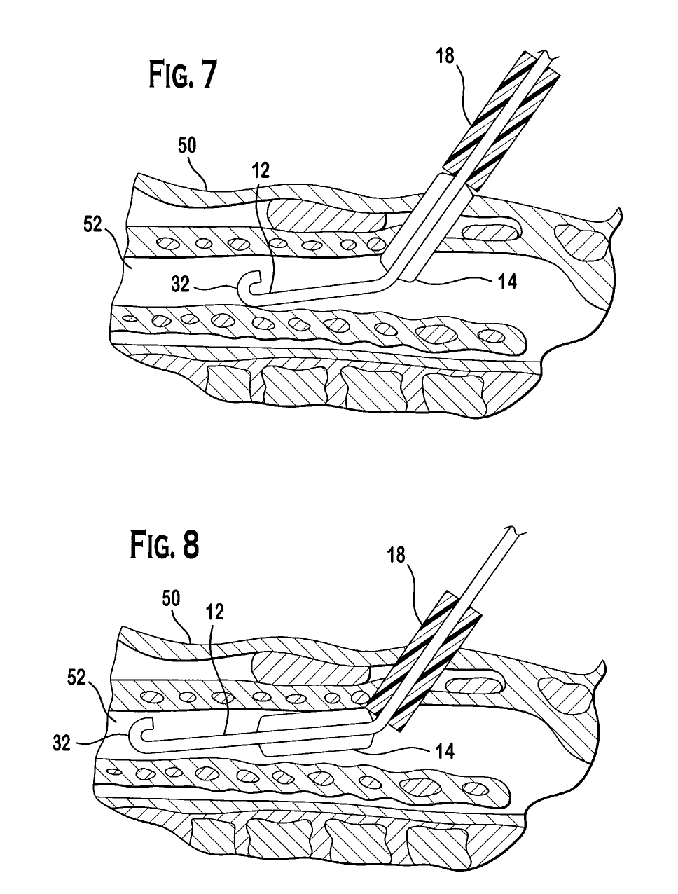

Shown in FIG. 4, the needle 16 is predisposed in the catheter 12 such that the pointed tip 46 of the needle 16 extends beyond the second catheter end 26 when the attachment receiving portion 44 is engaged with the needle attachment portion 30. The rigidity of the needle 16 causes the flexible end portion 32 to remain essentially straight for insertion of the pointed tip 46 of the needle 16 through the tracheal wall 50. As the needle 16 is inserted, the needle 16 punctures the skin adjacent to the tracheal wall 50 to create an opening. The pointed tip 46 of the needle 16 is inserted until the flexible end portion 32 of the second catheter end 28 is received in a tracheal passage 52. The needle 16 is then withdrawn from the catheter 12, leaving the flexible end portion 32 of the second catheter end 28 in the tracheal passage 52. As shown in FIG. 5, when the needle 16 is withdrawn, the flexible end portion 32 of the catheter 12 automatically curls to return to an essentially j-shape. Shown in FIGS. 6 and 7, the flexible end portion 32 is advanced inside the tracheal passage 52 until the inflatable balloon member 14 is received in the tracheal wall 50. Because the flexible end portion 32 has an essentially j-shape, advancement of the catheter 12 may be performed without damaging the interior of the tracheal wall 50.

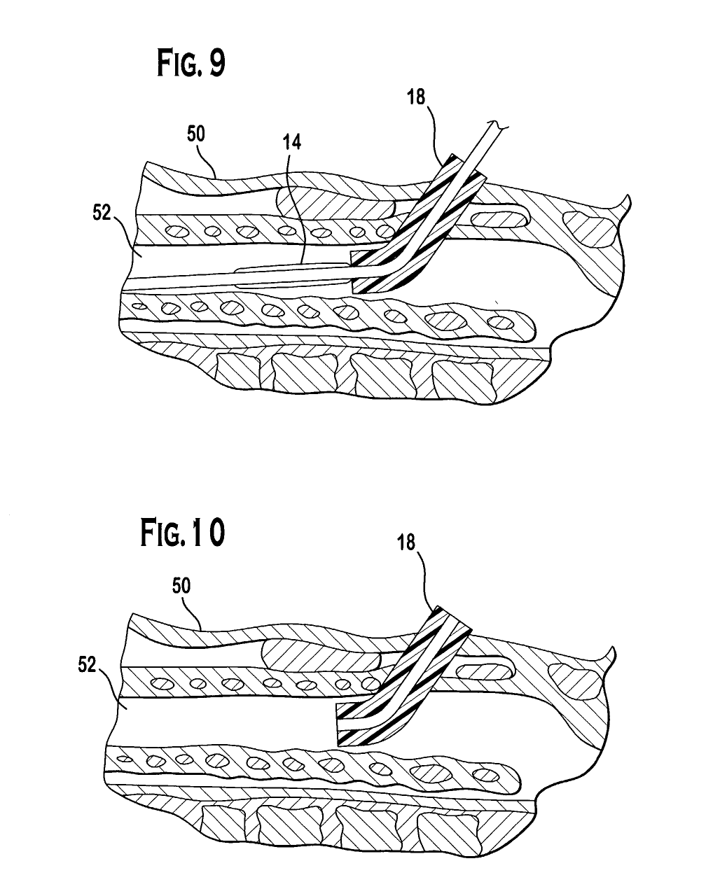

The conventional dilator (not shown) is activated to provide a fluid supply through a conventional dilator tube (not shown) engaged with the dilator attachment portion 24. The fluid supply is received in the second catheter end 28 and directed into the inflatable balloon member 14. Shown in FIG. 7, the inflatable balloon member 14 expands to dilate a portion of the tracheal wall 50 to form a passageway. After the inflatable balloon member 14 is inflated, the tubular member 18 is advanced along the catheter 12 toward the first balloon end 36, as shown in FIG. 8. The tubular member 18 contacts the first balloon end 36 and advances the first balloon end 36 into the tracheal passage 52 as the tubular member 18 is received in the passageway. As shown in FIGS. 9 and 10, the balloon member 14 is then deflated and the catheter 12 is withdrawn from the tubular member 18, leaving the tubular member 18 in the tracheal wall 50. Although the inflatable balloon member 14 is illustrated as being inflated when the tubular member 18 is advanced into the pasageway, it is well understood and appreciated by those skilled in the art that the inflatable balloon member 14 may also be deflated before advancement of the tubular member 18 into the passageway to obtain a substantially similar result.

While the device 10 is illustrated as being used in conjunction with the formation of a passageway in a tracheal wall for insertion of a tracheal tube, the device 10 may be used to form a passageway in any body tissue in a substantially similar manner. For example, the device 10 may be used in conjunction with other body cavities for insertion of other tubular members, such as chest tubes, gastrectomy tubes, trocars for laprascopic surgery and intra-abdominal drains. Further, although the attachment part is illustrated as formed with a needle attachment portion and a dilator attachment portion, the attachment part may be formed with a single attachment portion that receives the needle and attaches the conventional dilator such that after the needle is withdrawn from the single attachment portion of the catheter, the conventional dilator tube of the conventional dilator is attached to the single attachment portion to supply fluid to the inflatable balloon member.

FIGS. 11 and 12 show the second embodiment of the percutaneous medical insertion device 100. The device 100 comprises a central line or catheter 112, a one-way valve 114, a needle 116 and a covering mechanism 118. The catheter 112 has a first catheter end 126 connected to a medical fluid supply, and a second catheter end 128 having a flexible atraumatic end portion 120 that curls into an essentially j-shape (shown in phantom in FIG. 11) when the needle 116 is removed.

The individual components of the second embodiment of the device 100 will now be described in greater detail. Shown in FIG. 12, the catheter 112 comprises a flexible hollow tubular member extending from the first catheter end 126 to the second catheter end 128. The first catheter end 126 has parts of infusion with the possibility of several lumen for connection to a medical fluid supply, such as an in intravenous (IV) bag. The second catheter end 128 has the flexible end portion 120. The flexible end portion 120 essentially curls at the end of the catheter 112 in an essentially j-shape. At a base of the curl, the flexible end portion 120 has an essentially small fluid opening 122 for exit of fluid from the catheter 112 into the cavity. Positioned approximately 4 cm from the second end 128 is a slit 124. The slit 124 forms an opening when the catheter 112 is bent at an angle of essentially 90 degrees. The slit 124 is large enough that when the catheter 112 is bent at an angle of essentially 90 degrees, the needle 116 may be easily inserted into the catheter 112 and removed from the catheter 112.

Shown in FIG. 11, the needle 116 may be a hollow conventional surgical needle having a diameter smaller than the internal diameter of the catheter 112. The needle 116 has a syringe 132 and an essentially pointed tip 134 positioned opposite the syringe 132. The length of the needle 116 is such that the pointed tip 134 extends beyond the second end 128 of the catheter 112 when the needle 116 is removeably received in the catheter 112.

A valve 114 is positioned between the slit 124 and the flexible end portion 120. The one way valve 114 is positioned in the catheter 112 to prevent air emboli from occuring. The valve 114 may be any conventional one way valve that assists in the prevention of air emboli.

Positioned between the slit 124 and the first catheter end 126 is a moveable covering 118. The covering 118 is positioned about the outer circumference of the catheter 112. The covering 118 is moveably positioned adjacent to the slit 124 when the needle 116 is removed from the catheter 112 to prevent the excretion of fluids. The covering may be made of a thin plastic or any other pliable material. Alternatively, the covering 118 may be a separate piece attached to the outer circumference of the catheter 112 adjacent to the slit 124 after the needle 116 is removed.

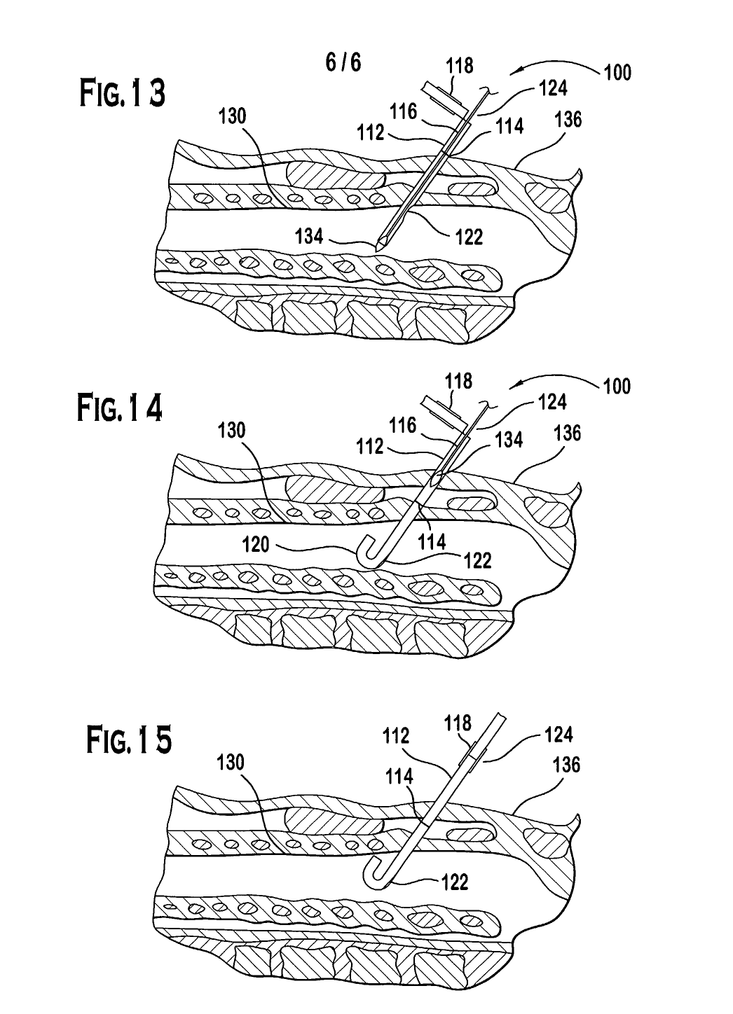

The method for placing the catheter 112 into a central vein 130 using the device 100 will now be described with reference to FIGS. 13 through 15. Although the device 100 is illustrated as positioning a central line or catheter 112 into a vein 130, such as a sub-clavian, internal jugular, or femoral vein, to allow for administration of large volumes of fluid, supplemental nutrition, blood draws, and pressure measurement, it will be understood and appreciated by those skilled in the art that the device 100 may also be used in conjunction with other medical applications, such as in abdominal or thoracic cavity procedures.

Shown in FIG. 13, the catheter 112 is bent at essentially 90 degrees proximate the slit 124 to form an opening for insertion of the pointed tip 134 of the needle 116. The needle 116 is inserted into the catheter 112 such that the pointed tip 134 of the needle 116 extends beyond the catheter second end 128. The rigidity of the needle 116 causes the flexible end portion 120 to remain essentially straight for insertion of the pointed tip 134 through a vein 130. As the needle 116 is inserted, the needle 116 punctures the skin 136 and the vein 130 to create an opening. The pointed tip 134 of the needle 116 is inserted until the flexible end portion 120 is received in the vein 130. Blood is then aspirated by the syringe 132 to confirm correct placement of the catheter 112 in the vein 130. The catheter 112 is slightly advanced while keeping the needle 116 still to allow the flexible end portion 120 of the catheter 112 to automatically curl to the essentially j-shape within the vein 130, as shown in FIG. 14. Blood is then aspirated by the syringe 132 to re-confirm correct placement of the catheter 112 in the vein 130 once the flexible end portion 120 is curled. The needle 116 is then withdrawn from the catheter 112 through the slit 124, leaving the flexible end portion 120 of the second end 128 in the vein 130. As the needle 116 is withdrawn, the valve 114 is activated to prevent the entry of air into the vein 130. After withdrawing the needle 116, the catheter 112 in positioned in an essentially straight position, and the covering 118 is advanced over the slit 124 to prevent escape of any fluids, as shown in FIG. 5. The catheter 112 is then advanced safely within the vein 130. Because the flexible end portion 120 has an essentially j-shape, advancement of the catheter 112 may be performed without damaging the interior of the vein 130. Fluid is then fed from the medical fluid supply through the first catheter end 126 and into the catheter 112. The fluid is easily administered into the vein 130 through the fluid opening 122 positioned at the base of the curve of the flexible end portion 120.

While the device 100 is illustrated as being used in conjunction with the positioning of a central line or a catheter into a vein, the device 100 may be used to position a central line or catheter in a wide variety of medical applications in a substantially similar manner. Further, although the catheter is illustrated as having a slit for removeably receiving the needle, the needle may be received in the first end of the catheter. After withdrawing the needle from the catheter, a conventional medical tube connected to the medical fluid supply may be attached to the first end of the catheter.

The foregoing illustrates some of the possibilities for practicing the invention. Many other embodiments are possible within the scope and spirit of the invention. It is, therefore, intended that the foregoing description be regarded as illustrative rather than limiting, and that the scope of the invention is given by the appended claims together with their full range of equivalents.

What is claimed is:

1. A percutaneous medical insertion device, comprising:
a catheter having a flexible end portion and an opening that removeably receives a needle, the flexible end portion is essentially straight when the needle is positioned in the flexible end portion and the flexible end portion essentially curls when the needle is removed from the flexible end portion.

2. The device of claim 1, wherein the flexible end portion curls into an essentially j-shape.

3. The device of claim 1, further comprising a valve positioned between the flexible end portion and the opening.

4. The device of claim 1, further comprising a covering positioned adjacent to the opening that moveably slides to cover the opening.

5. The device of claim 1, wherein the opening is a slit formed between a first end of the catheter and a second end of the catheter, the first end attached to a fluid supply and the second end having the flexible end portion.

6. The device of claim 1, wherein a base of the curl has an aperture for administration of fluid.

7. The device of claim 1, further comprising an inflatable balloon member disposed between the opening and the flexible end portion and a tubular member disposed about the catheter.

8. The device of claim 1, wherein the needle has a tip that extends beyond the flexible end portion.

9. The device of claim 8, wherein the needle has an attachment receiving portion distal from the tip and the catheter has an attachment part for engaging the attachment receiving portion.

10. The device of claim 1, wherein the catheter has an attachment portion for engaging a tube.

11. A percutaneous medical insertion device comprising:
a catheter having an inflatable balloon member disposed between a first catheter end and a second catheter end;
a needle removeably disposed in the catheter having a tip that extends beyond the second catheter end;
the second catheter end having a flexible end portion that curls into an essentially j-shape when the needle is removed from the catheter; and
a tubular member disposed about the catheter between the first catheter end and the inflatable balloon member.

12. The device of claim 8, wherein the needle has an attachment receiving portion distal from the tip and the first catheter end has an attachment part having a needle attachment portion for engaging the attachment receiving portion of the needle and a dilator attachment portion for engaging a dilator tube.

13. The device of claim 9, wherein the catheter has a length of essentially 17 cm.

14. The device of claim 10, wherein the inflatable balloon member has a length of essentially 3 cm and a first balloon end positioned essentially 8 cm from the first catheter end and a second balloon end positioned essentially 6 cm from the second catheter end.

15. The device of claim 9, wherein the inflatable balloon member is positioned essentially 6 cm from the second catheter end.

16. The device of claim 8, wherein the first catheter end has an attachment portion for engaging a dilator tube.

17. A percutaneous medical insertion device, comprising:
a catheter having a first end and a second end;
a needle removeable disposed in the catheter that is received in a slit between the first end and the second end;
the second end having a flexible end portion that curls when the needle is removed from the catheter; and
a valve positioned between the slit and the second end.

18. The device of claim 17, wherein the flexible end portion curls into an essentially j-shape.

19. The device of claim 17, further comprising a covering positioned adjacent to the opening that moveably slides to cover the opening.

20. The device of claim 17, wherein a base of the curl has an opening for administration of fluid received in the first end of the catheter.

21. A percutaneous method for positioning a central line or tubular member in the body, comprising;
percutaneously inserting a tip end of a needle removeably disposed in a catheter through a wall of an interior cavity or a vein into the interior cavity or the vein; and
withdrawing the needle from the catheter leaving the catheter disposed in the interior cavity or the vein.

22. The method of claim 21, further comprising:
advancing the catheter until an inflatable balloon member is received in the wall of the interior cavity; and
dilating the inflatable balloon member to form a passageway in the wall of the interior cavity.

23. The method of claim 22, further comprising:
advancing a tubular member into the wall of the interior cavity such that the inflatable balloon member is received in the interior cavity;
deflating the inflatable balloon member; and
withdrawing the inflatable balloon member and the catheter from the interior cavity leaving the tubular member in the wall of the interior cavity.

24. The method of claim 23, wherein the tubular member has an external diameter smaller than the diameter of the inflatable balloon member when the inflatable balloon member is dilated.

25. The method of claim 23, wherein the wall is a tracheal wall, the interior cavity is the interior of the tracheal wall and the tubular member is a tracheal tube.

26. The method of claim 21, further comprising:
aspirating fluid with a syringe to confirm correct positioning of the catheter; and
covering an opening that receives the needle when the needle is withdrawn from the catheter.

27. The method of claim 26, further comprising:
advancing the catheter about the needle; and
activating a valve.

28. The method of claim 27, wherein the catheter has a flexible end portion that curls into an essentially j-shape when the catheter is advanced about the needle to protect the interior of the interior cavity or the vein.

29. The method of claim 21, wherein the catheter has a flexible end portion that curls into an essentially j-shape when the needle is removed from the catheter to protect the interior of the interior cavity or the vein.
